(19) United States
(12) Patent Application Publication
(10) Pub. No.: US 2012/0261321 A1
(43) Pub. Date: Oct. 18, 2012

(54) SEPARATION MEMBRANE, METHOD FOR MANUFACTURING THE SAME, AND FORWARD OSMOSIS DEVICE INCLUDING THE SAME

(75) Inventors: Jung Im Han, Yongin-si (KR); Sung Soo Han, Hwaseong-si (KR); Hyo Kang, Seoul (KR)

(73) Assignee: SAMSUNG ELECTRONICS CO., LTD., Suwon-si (KR)

(21) Appl. No.: 13/448,618

(22) Filed: Apr. 17, 2012

(30) Foreign Application Priority Data
Apr. 18, 2011 (KR) 10-2011-0035868
Mar. 22, 2012 (KR) 10-2012-0029340

(51) Int. Cl. B01D 71/12; A61M 1/22; B01D 69/12; C08B 13/00; B01D 69/06

(52) U.S. Cl. 210/195.2; 536/66; 264/48; 210/489

(57) ABSTRACT

Example embodiments relate to a separation membrane including at least one polymer including a structural unit represented by the following Chemical Formula 1,

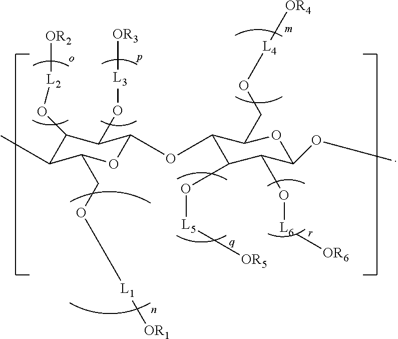

Chemical Formula 1 may be as described in the detailed description. Example embodiments also relate to a forward osmosis device including the separation membrane, methods of preparing the polymer of the separation membrane, and methods of manufacturing the separation membrane.

### CROSS-REFERENCE TO RELATED APPLICATIONS

This application claims priority under 35 U.S.C. §119 to Korean Patent Application Nos. 10-2011-0035868 and 10-2012-0029340, filed in the Korean Intellectual Property Office on Apr. 18, 2011 and Mar. 22, 2012, respectively, the entire contents of each of which are incorporated herein by reference.

### BACKGROUND

1. Field

Example embodiments herein relate to a separation membrane, a method of manufacturing the same, and a forward osmosis device including the separation membrane.

2. Description of the Related Art

Interest in forward osmosis (FO) has grown with the increasing demand for a lower energy consumption and higher efficiency membrane. Forward osmosis, like reverse osmosis, requires a separation membrane that is capable of filtering a solute by inducing osmotic pressure. However, unlike reverse osmosis, forward osmosis uses a concentration difference instead of a pressure difference to separate materials. Thus, a forward osmosis process may be operated under relatively low pressure or even without pressure. According to a recent study, energy consumption per ton of water produced by reverse osmosis in sea water desalination is about 3-5 kWh, while energy consumption per ton may be lowered to about 1 kWh using forward osmosis.

Internal concentration polarization of a membrane is an important factor affecting the performance of a forward osmosis system. Concentration polarization refers to a phenomenon in which the concentrations of materials around the surface and inside of a separation membrane vary from the surrounding environment during the process of separating water from a solution. Therefore, operation performance of a separation membrane may fall below theoretically calculated values. Concentration polarization occurring around the separation membrane surface is referred to as external concentration polarization, which may be solved by controlling the operation conditions of the separation membrane. However, concentration polarization occurring inside of a separation membrane may be relatively difficult to solve.

If a separation membrane commonly used for a reverse osmosis process is used for a forward osmosis process, significant concentration polarization may occur. In the forward osmosis process, chemical properties of the separation membrane are also an important factor affecting performance, as well as the structure of the separation membrane. In the reverse osmosis process, since movement of water passing a separation membrane occurs by pressure, the chemical properties of the support are not a critical parameter with regard to membrane permeation flow rate. However, in the forward osmosis process, since water permeation spontaneously occurs by an osmotic pressure difference, chemical properties of the support, i.e., hydrophilicity, largely influence the permeation flow rate.

### SUMMARY

Various embodiments relate to a separation membrane including a polymer having a relatively high strength, porosity, and hydrophilicity.

Various embodiments relate to a method of manufacturing the polymer.

Various embodiments relate to a method of manufacturing the separation membrane.

Various embodiments relate to a forward osmosis device including the separation membrane.

According to a non-limiting embodiment, a separation membrane may include at least one polymer including a structural unit represented by the following Chemical Formula 1.

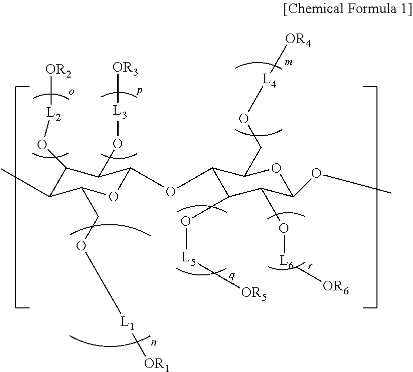

In the above Chemical Formula 1,

R₁ to R₆ may each independently be a hydrogen, a substituted or unsubstituted C1 to C30 alkyl group, a substituted or unsubstituted C3 to C30 cycloalkyl group, a substituted or unsubstituted C2 to C30 heterocycloalkyl group, a substituted or unsubstituted C6 to C30 aryl group, a substituted or unsubstituted C2 to C30 heteroaryl group, a substituted or unsubstituted C7 to C30 alkylaryl group, a substituted or unsubstituted C7 to C30 arylalkyl group, or —COR₇,

R₇ may be a substituted or unsubstituted C1 to C30 alkyl group, a substituted or unsubstituted C3 to C30 cycloalkyl group, a substituted or unsubstituted C2 to C30 heterocycloalkyl group, a substituted or unsubstituted C6 to C30 aryl group, a substituted or unsubstituted C2 to C30 heteroaryl group, a substituted or unsubstituted C7 to C30 alkylaryl group, or a substituted or unsubstituted C7 to C30 arylalkyl group,

provided that at least one of R₁ to R₃ and at least one of R₄ to R₆ are each independently the same or different and are —COR₇,

at least one of R₁ to R₃ and at least one of R₄ to R₆ are each independently the same or different, and are a substituted or unsubstituted C1 to C30 alkylene group, a substituted or unsubstituted C3 to C30 cycloalkylene group, a substituted or unsubstituted C2 to C30 heterocycloalkylene group, a substituted or unsubstituted C6 to C30 arylene group, a substituted or unsubstituted C2 to C30 heteroarylene group, a substituted or unsubstituted C7 to C30 alkylarylene group, or a substituted or unsubstituted C7 to C30 arylalkylene group,

L₁ to L₆ may each independently be a substituted or unsubstituted C1 to C30 alkylene group, a substituted or unsubstituted C3 to C30 cycloalkylene group, a substituted or unsubstituted C2 to C30 heterocycloalkylene group, a substituted or unsubstituted C6 to C30 arylene group, a substituted or unsubstituted C2 to C30 heteroarylene group, a substituted or unsubstituted C7 to C30 alkylarylene group, or a substituted or unsubstituted C7 to C30 arylalkylene group,

n and m may each independently be an integer ranging from 0 to 150, the sum of n and m being at least 1, and

o, p, q, and r may each independently be an integer ranging from 0 to 100.

The polymer may have a first degree of substitution (DS) by R₁ to R₆ of an alkyl group, a cycloalkyl group, a heterocycloalkyl group, an aryl group, a heteroaryl group, an alkylaryl group, or an arylalkyl group of about 1 to about 2 per anhydrous glucose unit, and a second degree of substitution by R₁ to R₆ of —COR₇ in the above Chemical Formula 1 of about 1 to about 2 per anhydrous glucose unit.

The polymer may have a weight average molecular weight of about 20,000 to about 800,000.

The separation membrane may have a contact angle with regard to water of about 50° to about 65°.

The separation membrane may be a single membrane formed of a skin layer and a porous layer, wherein the skin layer has higher density than the porous layer.

The separation membrane may be insoluble in water, and soluble in an organic solvent selected from acetone, acetic acid, methanol, isopropanol, 1-methoxy-2-propanol, trifluoroacetic acid (TFA), tetrahydrofuran (THF), pyridine, methylene chloride, dimethyl formamide (DMF), dimethyl acetamide (DMAC), N-methyl-2-pyrrolidone (NMP), terpineol, 2-butoxyethylacetate, 2-(2-butoxyethoxy)ethylacetate, and a combination thereof.

The separation membrane may be a microfiltration membrane, an ultrafiltration membrane, a nanofiltration membrane, a reverse osmotic membrane, or a forward osmotic membrane.

According to yet another non-limiting embodiment, a method of preparing a polymer including a structural unit represented by the following Chemical Formula 1 is provided. The method may include etherifying a cellulose compound to obtain a cellulose ether compound having at least one hydroxyl group, and esterifying the cellulose ether compound to obtain the polymer in the form of an esterified cellulose ether.

The cellulose ether compound having at least one hydroxyl group may be obtained by substituting hydrogen of at least one hydroxyl group (e.g. first hydroxyl group) of the cellulose compound with an alkyl group, a cycloalkyl group, a heterocycloalkyl group, an aryl group, a heteroaryl group, an alkylaryl group or an arylalkyl group to form an ether group; and substituting a hydrogen of at least one hydroxyl group (e.g., second hydroxyl group) of the cellulose compound with an alkyl group, a cycloalkyl group, a heterocycloalkyl group, an aryl group, a heteroaryl group, an alkylaryl group or an arylalkyl group that includes at least one hydroxyl group (e.g., third hydroxyl group). The method may further include repeatedly substituting a hydrogen of the hydroxyl group (e.g., third hydroxyl group) introduced by the above substitution with an alkyl group, a cycloalkyl group, a heterocycloalkyl group, an aryl group, a heteroaryl group, an alkylaryl group or an arylalkyl group that includes at least one hydroxyl group (e.g., fourth hydroxyl group) so as to form a moiety of —(O-L₁)ₙ-, —(O-L₂)ₒ-, —(O-L₃)ₚ-, —(O-L₄)ₘ-, —(O-L₅)_{q}-, or —(O-L₆)ᵣ- in the structure of the above Chemical Formula 1.

The hydrogen of the hydroxyl group of the cellulose ether compound having at least one hydroxyl group may be substituted with a —COR₇ group to esterify the cellulose ether compound so as to obtain an esterified cellulose ether.

The polymer may have a weight average molecular weight of about 20,000 to about 800,000.

According to yet another non-limiting embodiment, a method of manufacturing a separation membrane is provided. The method may include preparing a polymer solution including at least one polymer including a structural unit represented by the above Chemical Formula 1, and an organic solvent; casting the polymer solution on a substrate; and immersing the substrate casted with the polymer solution in a non-solvent to form a skin layer and a porous layer.

The polymer solution may include about 5 to about 30 wt % of the polymer, about 0 to about 10 wt % of a pore forming agent, and about 50 to about 95 wt % of the organic solvent.

The substrate may be a glass plate or a polyester non-woven fabric.

The polymer solution may be cast on the substrate to a thickness of about 25 μm to about 300 μm.

The pore forming agent may include polyvinylpyrrolidone, polyethylene glycol, polyethyloxazoline, glycerol, ethylene glycol, diethylene glycol, ethanol, methanol, acetone, phosphoric acid, acetic acid, propanoic acid, lithium chloride, lithium nitrate, lithium perchlorate, or a combination thereof.

The organic solvent may include acetone, acetic acid methanol, 1-methoxy-2-propanol, 1,4-dioxane with a boiling point of less than about 120° C., N-methyl-2-pyrrolidone (NMP), dimethyl acetamide (DMAC), dimethyl formamide (DMF) with a boiling point of about 150° C. to about 300° C., or a combination thereof.

According to still another non-limiting embodiment, a forward osmosis device is provided. The forward osmosis device may include a feed solution including impurities to be purified; an osmosis draw solution having higher osmotic pressure than the feed solution; the above-explained separation membrane positioned so that one side contacts the feed solution and the other opposing side contacts the osmosis draw solution; a recovery system for separating a draw solute from the osmosis draw solution; and a connector for reintroducing the draw solute of the osmosis draw solution separated by the recovery system into the osmosis draw solution contacting the separation membrane.

The forward osmosis device may further include a means (e.g., treatment portion) for producing treated water from the rest of the osmosis draw solution including the water that has passed through the semi-permeable separation membrane by osmotic pressure from the feed solution to the osmosis draw solution, from which draw solute has been separated by the recovery system.

### BRIEF DESCRIPTION OF THE DRAWINGS

FIG. 1 is a schematic view illustrating a contact angle formed between the surface of a substrate and a droplet according to a non-limiting embodiment.

FIGS. 2A-2B are schematic cross-sectional views of a separation membrane according to a non-limiting embodiment.

FIG. 3 is a schematic view of a forward osmosis device according to a non-limiting embodiment.

FIGS. 4A-4B are SEM photographs of a separation membrane according to a non-limiting embodiment.

FIG. 5 is a graph showing the measurement results of contact angles and tensile strength of various examples and comparative examples.

FIG. 6 is a graph showing water permeation flow rates according to a non-limiting embodiment.

### DETAILED DESCRIPTION

This disclosure will be described more fully hereinafter in the following detailed description. It should be understood that this disclosure may be embodied in many different forms and is not be construed as limited to the embodiments set forth herein.

As used herein, when a definition is not otherwise provided, the term “substituted” may refer to one substituted with a C1 to C30 alkyl group; a C1 to 010 alkylsilyl group; a C3 to C30 cycloalkyl group; a C6 to C30 aryl group; a C2 to C30 heteroaryl group; a C1 to C10 alkoxy group; a fluoro group, a C1 to 010 trifluoroalkyl group such as a trifluoromethyl group; or a cyano group.

As used herein, when a definition is not otherwise provided, the prefix “hetero” may refer to one including 1 to 3 heteroatoms selected from N, O, S, and P, with the remaining structural atoms in a compound or a substituent being carbon atoms.

As used herein, when a definition is not otherwise provided, the term “combination thereof” refers to at least two substituents bound to each other by a linker, or at least two substituents condensed to each other.

As used herein, when a definition is not otherwise provided, the term “alkyl group” may refer to a “saturated alkyl group” without an alkenyl group or an alkynyl group, or an “unsaturated alkyl group” including at least one of an alkenyl group or an alkynyl group. The term “alkenyl group” may refer to a substituent in which at least two carbon atoms are bound in at least one carbon-carbon double bond, and the term “alkynyl group” refers to a substituent in which at least two carbon atoms are bound in at least one carbon-carbon triple bond. The alkyl group may be a branched, linear, or cyclic alkyl group.

The alkyl group may be a linear or branched C1 to C20 alkyl group, and more specifically a C1 to C6 alkyl group, a C7 to 010 alkyl group, or a C11 to C20 alkyl group.

For example, a C1-C4 alkyl may have 1 to 4 carbon atoms, and may be selected from the group consisting of methyl, ethyl, propyl, iso-propyl, n-butyl, iso-butyl, sec-butyl, and t-butyl.

Examples of the alkyl group include a methyl group, an ethyl group, a propyl group, an isopropyl group, a butyl group, an isobutyl group, a t-butyl group, a pentyl group, a hexyl group, an ethenyl group, a propenyl group, a butenyl group, a cyclopropyl group, a cyclobutyl group, a cyclopentyl group, a cyclohexyl group, and the like.

The term “aromatic group” may refer a substituent including a cyclic structure where all elements have p-orbitals which form conjugation. For example, an aryl group and a heteroaryl group may be utilized.

The term “aryl group” may refer to a monocyclic or fused ring-containing polycyclic (i.e., rings sharing adjacent pairs of carbon atoms) groups. When the heteroaryl group is a fused ring, each ring may include 1 to 3 heteroatoms.

The separation membrane according to a non-limiting embodiment may include at least one polymer including a structural unit represented by the following Chemical Formula 1.

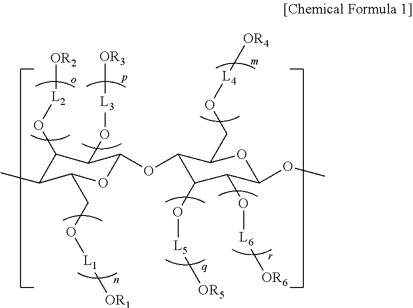

In the above Chemical Formula 1,

R₁ to R₆ may each independently be a hydrogen, a substituted or unsubstituted C1 to C30 alkyl group, a substituted or unsubstituted C3 to C30 cycloalkyl group, a substituted or unsubstituted C2 to C30 heterocycloalkyl group, a substituted or unsubstituted C6 to C30 aryl group, a substituted or unsubstituted C2 to C30 heteroaryl group, a substituted or unsubstituted C7 to C30 alkylaryl group, a substituted or unsubstituted C7 to C30 arylalkyl group, or —COR₇,

R₇ may be a substituted or unsubstituted C1 to C30 alkyl group, a substituted or unsubstituted C3 to C30 cycloalkyl group, a substituted or unsubstituted C2 to C30 heterocycloalkyl group, a substituted or unsubstituted C6 to C30 aryl group, a substituted or unsubstituted C2 to C30 heteroaryl group, a substituted or unsubstituted C7 to C30 alkylaryl group, or a substituted or unsubstituted C7 to C30 arylalkyl group,

provided that at least one of R₁ to R₃ and at least one of R₄ to R₆ are each independently the same or different and are —COR₇, and

at least one of R₁ to R₃ and at least one of R₄ to R₆ are each independently the same or different, and are a substituted or unsubstituted C1 to C30 alkylene group, a substituted or unsubstituted C3 to C30 cycloalkylene group, a substituted or unsubstituted C2 to C30 heterocycloalkylene group, a substituted or unsubstituted C6 to C30 arylene group, a substituted or unsubstituted C2 to C30 heteroarylene group, a substituted or unsubstituted C7 to C30 alkylarylene group, or a substituted or unsubstituted C7 to C30 arylalkylene group,

L₁ to L₆ may each independently be a substituted or unsubstituted C1 to C30 alkylene group, a substituted or unsubstituted C3 to C30 cycloalkylene group, a substituted or unsubstituted C2 to C30 heterocycloalkylene group, a substituted or unsubstituted C6 to C30 arylene group, a substituted or unsubstituted C2 to C30 heteroarylene group, a substituted or unsubstituted C7 to C30 alkylarylene group, or a substituted or unsubstituted C7 to C30 arylalkylene group,

n and m may each independently be an integer ranging from 0 to 150, and

o, p, q, and r may each independently be an integer ranging from 0 to 100.

Since the polymer is insoluble in water, it is relatively easy to manufacture a separation membrane. Higher hydrophilicity and higher strength may also be realized. In an example of a structural unit represented by Chemical Formula 1, the sum of n and m is at least 1. Additionally, in another example of a structural unit represented by Chemical Formula 1, the sum of o and p is at least 1. Furthermore, in another example of a structural unit represented by Chemical Formula 1, the sum of q and r is at least 1.

The polymer may be insoluble in water due to a hydrophobic ester group. The polymer may also exhibit hydrophilicity because of a cellulose backbone. The degree of hydrophilicity as well as solubility in a specific solvent may be adjusted by appropriately controlling the kind of substituents such as an alkyl group or an aryl group, and the like, and the substitution degree. As a result, the polymer may be prepared so as to be insoluble in water, while being soluble in a specific organic solvent selected from acetone, acetic acid, methanol, isopropanol, 1-methoxy-2-propanol, trifluoroacetic acid (TFA), tetrahydrofuran (THF), pyridine, methylene chloride, dimethyl formamide (DMF), dimethyl acetamide (DMAC), N-methyl-2-pyrrolidone (NMP), terpineol, 2-butoxyethylacetate, 2 (2-butoxyethoxy)ethylacetate, and a combination thereof.

The polymer may be subjected to a process such as solvent casting, wet spinning, dry spinning, and the like using the above properties. The polymer may also be applied to a melt process such as injection and melt spinning, and the like, because of its melting point.

For example, the polymer may have a first degree of substitution (DS) by R₁ to R₆ of an alkyl group, a cycloalkyl group, a heterocycloalkyl group, an aryl group, a heteroaryl group, an alkylaryl group, or an arylalkyl group, of about 1 to about 2 per anhydrous glucose unit. The polymer may also have a second degree of substitution by R₁ to R₆ of —COR₇ in the above Chemical Formula 1 of about 1 to about 2 per anhydrous glucose unit.

The degree of substitution (DS) may refer to an average number of substituted hydroxyl groups per anhydrous glucose unit. Since a maximum of 3 hydroxyl groups exist per anhydrous glucose unit, the theoretical maximum degree of substitution with a mono-functional substituent is 3.

Since the polymer may be prepared so as to have a relatively high molecular weight by the subsequently mentioned manufacturing method, a relatively high strength may be realized. If the porosity of a separation membrane increases, the strength may decrease. Thus, the strength may be compensated by preparing a polymer with a higher molecular weight. Since the polymer may be prepared with a relatively high molecular weight, higher strength may be realized, and thus the membrane may be prepared so as to have higher porosity. For example, the polymer may have a weight average molecular weight of about 20,000 to about 800,000. For another example, the polymer may have a weight average molecular weight of about 100,000 to about 200,000 considering a membrane forming property such as viscosity and the like. If the weight average molecular weight is about 200,000, a polymer solution with a polymer concentration of about 7% to about 10% may be prepared and used. If the weight average molecular weight is about 500,000 or more, polymer solubility tends to decrease. When the polymer has a molecular weight within the above range, it may have strength suitable for manufacturing a separation membrane.

According to another non-limiting embodiment, a method of preparing the polymer is provided. The method of preparing the polymer includes etherifying a cellulose compound to obtain a cellulose ether compound having at least one hydroxyl group, and esterifying the cellulose ether compound to obtain an esterified cellulose ether.

Hereinafter, a method for preparing the polymer according to a non-limiting embodiment will be explained in further detail.

First, a hydrogen of at least a first hydroxyl group of cellulose is substituted with an alkyl group, a cycloalkyl group, a heterocycloalkyl group, an aryl group, a heteroaryl group, an alkylaryl group or an arylalkyl group (hereinafter, referred to as ‘substituent’) to form an ether group.

A hydrogen of at least a second hydroxyl group of the cellulose is also substituted with an alkyl group, a cycloalkyl group, a heterocycloalkyl group, an aryl group, a heteroaryl group, an alkylaryl group or an arylalkyl group containing at least a third hydroxyl group (hereinafter, referred to as ‘substituent containing at least one hydroxyl group’).

The alkyl group or the alkyl group of the substituent containing at least one hydroxyl group may include a linear or branched type.

The hydrogen of the at least one hydroxyl group of the substituent containing at least one hydroxyl group may also be substituted with a substituent or a substituent containing at least one hydroxyl group. Thus, the hydrogen of the at least one hydroxyl group of the substituent containing at least one hydroxyl group may be repeatedly substituted to form a moiety of —(O-L₁)ₙ-, —(O-L₂)ₒ-, —(O-L₃)ₚ-, —(O-L₄)ₘ-, —(O-L₆)_{q}-, or —(O-L₆)ᵣ- in the structure of the above Chemical Formula 1.

In this way, a cellulose compound is primarily etherified, and thereby, a hydrogen bond of cellulose is broken, and the compound is converted into an amorphous structure. The synthesized cellulose ether has an amorphous structure, and the hydroxyl group included in the cellulose compound becomes a hydroxyl group having a desirable level of reactivity. Subsequently, the hydrogen of the hydroxyl group having the desirable level of reactivity is substituted with a —COR₇ group (this substitution reaction is referred to as esterification) to esterify the cellulose ether. Thus, an esterified cellulose ether is obtained.

According to the above preparation method, cellulose may be sequentially etherified and esterified. Consequently, the cellulose may be esterified without substantially decreasing the molecular weight. Namely, according to the above manufacturing method, there is no need to break a crystal structure of cellulose for esterification. Since a polar catalyst such as inorganic acid that is used to break a crystal structure of cellulose is not used, a main chain of cellulose may not be cut by a polar catalyst, thus allowing the attainment of an esterified cellulose ether having a higher molecular weight. A membrane manufactured with the esterified cellulose ether having a higher molecular weight may exhibit higher strength and greater durability while exhibiting hydrophilicity.

The polymer has hydrophilicity resulting from cellulose. The hydrophilicity may be adjusted by controlling the kind and degree of the substituents, wherein the degree of hydrophilicity of the separation membrane including the polymer may be measured by dropping a droplet on the surface of the separation membrane to measure a contact angle. For example, the degree of substitution by an ester group may be measured and controlled by titration.

The term “contact angle” used in the present specification is defined as follows.

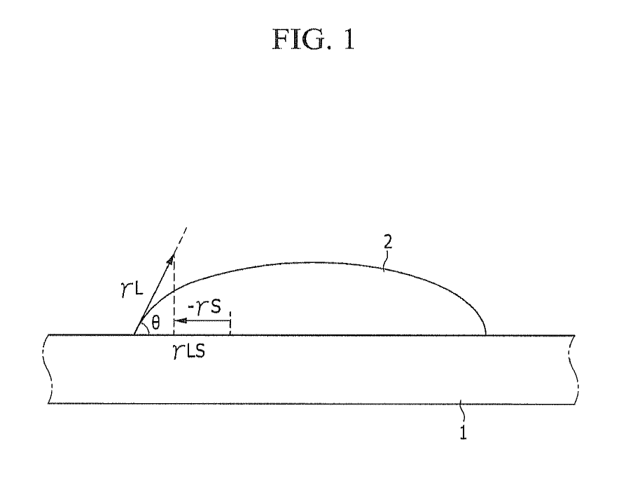

FIG. 1 is a schematic view illustrating a contact angle formed between the surface of a substrate and a droplet according to a non-limiting embodiment.

Generally, the shape of a bell-type droplet 2 existing on the surface of a substrate 1 may be defined as a contact angle (θ). The following Equation 1 (Young's Equation) is realized among the contact angle (θ), surface tension (γL) of a droplet, and surface energy (γS) of a substrate. In Equation 1, γLS denotes interface energy between the surface of the substrate 1 and the droplet 2.

cos θ=(γS−γLS)/γL[Equation 1]

In Equation 1, γLS decreases along with a decrease of γS, and when γS is decreased, it is generally known that the decrease amount of γLS is smaller than γS. Additional details regarding Young's Equation may be found in D. T. Kaelble, J. Adhesion, vol. 2 1970, pp. 66-81, the contents of which are incorporated herein by reference. Therefore, when the surface energy γS of the substrate 1 is decreased, the right side value of Equation 1 is decreased and the contact angle (θ) is increased. Therefore, the droplet 2 discharged onto the surface of the substrate 1 shrinks as time passes. Equation 1 may be represented by a vector as shown in FIG. 1.

In short, when the contact angle of the droplet 2 is relatively small, it means that the droplet 2 is spread relatively widely on the substrate 1, which means that the substrate 1 and the droplet 2 have a chemical attraction with each other.

For example, the contact angle of the separation membrane to water may be about 50° to about 65°. The contact angle increases by roughness when forming a separation membrane, and thus is largely influenced by the structure of pores. Therefore, a pore characteristic may be indirectly determined from the contact angle.

For a hydrophobic material, when water first enters into a relatively small pore, a relatively strong push with pressure or hydrophilic treatment is required. On the other hand, for a hydrophilic material, water may spontaneously enter into the pore by osmosis due to its wettability, thus reducing the generation of dead pores. Further, a hydrophobic material may generate a trap such as a bubble when operating the membrane, while a hydrophilic material is more advantageous in terms of mass transfer, thus reducing the generation of dead pores.

Since the separation membrane has improved hydrophilicity, water permeation resistance may be reduced.

The separation membrane may be manufactured as a single layer formed of a skin layer and a porous layer. For example, a single layer formed of a skin layer and a porous layer may be manufactured using the polymer by non-solvent induced phase separation (NIPS). For the details of the non-solvent induced phase separation, the subsequently explained manufacturing method of the separation membrane will be referred to.

In the separation membrane formed of a skin layer and a porous layer, for example, the ratio of the thickness of the skin layer to the thickness of the porous layer may be about 0.001 to about 0.1.

For example, the skin layer may have a thickness of about 0.1 μm to about 10 μm.

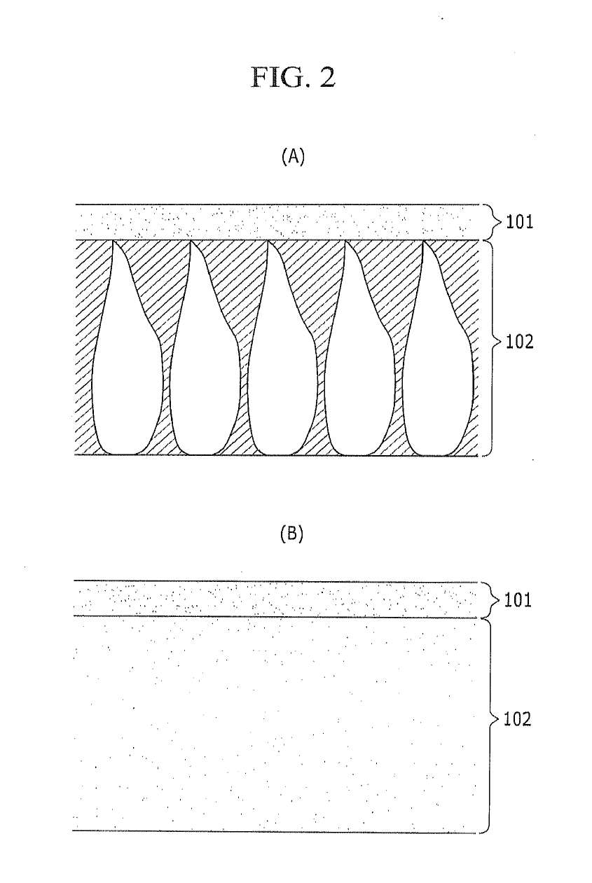

FIGS. 2A-2B are schematic cross-sectional views of a separation membrane according to a non-limiting embodiment. The separation membrane is in the form of a single layer formed of a skin layer and a porous layer, wherein a skin layer 101 having relatively high density and a porous layer 102 having a relatively low density are stacked. The porous layer may be realized so as to have various shapes of pore structures. The shape of the pores, porosity, and the like may be modified according to the type of the separation membrane. For example, a finger-like structure, a sponge-type structure, a finger-like/sponge type mixed structure, and the like may be enumerated.

FIG. 2(A) shows a case wherein the porous layer 102 is formed in a finger-like structure, and FIG. 2(B) shows a case wherein the porous layer 102 is formed in a sponge-type structure. The finger-like structure, the sponge-type structure, and the like may be manufactured by a known method without specific limitation. For example, the finger-like structure or the sponge type structure may be manufactured by non-solvent induced phase separation while varying the process conditions.

The characteristics of the structure of the pore forming the membrane may be indicated by tortuosity (τ) and porosity (ε).

The tortuosity (τ) of the membrane is a ratio of substantial path of water in the membrane to the thickness of the membrane. A tortuosity of 1 means that water may pass vertically through the membrane in the thickness direction without being disturbed. If a structure disturbing the movement of water exists in the membrane (e.g., if density of the membrane is relatively high), the tortuosity may increase. Thus, the lower limit of the tortuosity is 1. A unit of tortuosity does not exist, because it is a ratio. If the tortuosity increases, a structure factor increases.

The porosity (ε) of the membrane means a ratio of the pores to the internal volume of the membrane. Thus, porosity of 1 refers to that the inside of the membrane is empty. Density increases as the porosity decreases. If the porosity increases, factors disturbing the movement of water in the membrane decrease, and thus a structure factor decreases. A unit of porosity does not exist, because it is a ratio.

For example, the porous layer may have porosity (ε) of about 0.5 to about 0.99, and a tortuosity (τ) of about 1 to about 2.5.

According to yet another non-limiting embodiment, a method of manufacturing the separation membrane is provided.

The method of manufacturing the separation membrane may include preparing a polymer solution including at least one polymer including a structural unit represented by the following Chemical Formula 1, as well as an organic solvent; casting the polymer solution on a substrate; and immersing the substrate cast with the polymer solution in a non-solvent.

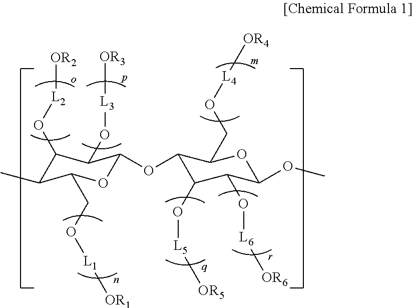

In the above Chemical Formula 1,

R₁ to R₆ may each independently be a hydrogen, a substituted or unsubstituted C1 to C30 alkyl group, a substituted or unsubstituted C3 to C30 cycloalkyl group, a substituted or unsubstituted C2 to C30 heterocycloalkyl group, a substituted or unsubstituted C6 to C30 aryl group, a substituted or unsubstituted C2 to C30 heteroaryl group, a substituted or unsubstituted C7 to C30 alkylaryl group, a substituted or unsubstituted C7 to C30 arylalkyl group, or —COR₇,

R₇ may be a substituted or unsubstituted C1 to C30 alkyl group, a substituted or unsubstituted C3 to C30 cycloalkyl group, a substituted or unsubstituted C2 to C30 heterocycloalkyl group, a substituted or unsubstituted C6 to C30 aryl group, a substituted or unsubstituted C2 to C30 heteroaryl group, a substituted or unsubstituted C7 to C30 alkylaryl group, or a substituted or unsubstituted C7 to C30 arylalkyl group,

provided that at least one of R₁ to R₃ and at least one of R₄ to R₆ are each independently the same or different and are —COR₇, and

at least one of R₁ to R₃ and at least one of R₄ to R₆ are each independently the same or different, and are a substituted or unsubstituted C1 to C30 alkylene group, a substituted or unsubstituted C3 to C30 cycloalkylene group, a substituted or unsubstituted C2 to C30 heterocycloalkylene group, a substituted or unsubstituted C6 to C30 arylene group, a substituted or unsubstituted C2 to C30 heteroarylene group, a substituted or unsubstituted C7 to C30 alkylarylene group, or a substituted or unsubstituted C7 to C30 arylalkylene group,

L₁ to L₆ may each independently be a substituted or unsubstituted C1 to C30 alkylene group, a substituted or unsubstituted C3 to C30 cycloalkylene group, a substituted or unsubstituted C2 to C30 heterocycloalkylene group, a substituted or unsubstituted C6 to C30 arylene group, a substituted or unsubstituted C2 to C30 heteroarylene group, a substituted or unsubstituted C7 to C30 alkylarylene group, or a substituted or unsubstituted C7 to C30 arylalkylene group,

n and m may each independently be an integer ranging from 0 to 150, and

o, p, q, and r may each independently be an integer ranging from 0 to 100.

The polymer solution may have viscosity of about 5 to about 100,000 cps under conditions of about 20° C. and about 20 rpm, as measured by a Brookfield viscometer. If the polymer solution has a viscosity within the above range, the mechanical strength of the polymer may be appropriate for manufacturing a general membrane.

Casting of the polymer solution on a substrate may be performed under relative humidity of about 65±5% and a temperature of about 25±1° C.

The immersing of the substrate casted with the polymer solution in a non-solvent may be performed by precipitating the substrate casted with the polymer solution in a non-solvent coagulation bath to form a membrane. Since the organic solvent in the polymer solution and a non-solvent are miscible, and the polymer is insoluble in a non-solvent, and if the substrate casted with the polymer solution is immersed in a non-solvent, a skin layer and a porous layer are produced to form a membrane.

Before immersing the substrate casted with the polymer solution in a non-solvent coagulation bath, evaporation may be further carried out, and the evaporation may be performed at about 20° C. to about 40° C., for about 1 minute to about 30 minutes.

As the coagulation bath, for example, distilled water may be used, and the temperature may be controlled to about 15° C. to about 50° C. The immersing time in the coagulation bath may be about 1 minute to about 30 minutes.

After immersing the substrate casted with the polymer solution in the non-solvent coagulation bath to form a membrane, the membrane may be annealed at about 50° C. to about 100° C.

The internal structure of the membrane and the structure of the pore may be varied by controlling the process conditions such as evaporation time and the heat treatment temperature and time. Under the process conditions within the above illustrated ranges, surface pores of the membrane may become smaller and the internal porosity may become larger.

A skin layer and a porous layer with a finger-like internal pore structure may be formed by the above separation membrane manufacturing method, and the porous layer may impart porosity to the membrane thus reducing or minimizing internal concentration polarization. The finger-like structure is advantageous for securing a desirable permeation flow rate. The separation membrane may be applied for salinity gradient energy using osmotic pressure, as well as in the water treatment field such as for water purification, waste water treatment and reuse, sea water desalination, and the like.

The details of the polymer may be as described above.

In the separation membrane manufacturing method, the polymer solution may include about 5 to about 30 wt % of the polymer, about 0 to about 10 wt % of a pore forming agent, and about 60 to about 95 wt % of the organic solvent. The above range is suitable for manufacturing a separation membrane using non-solvent induced phase separation (NIPS).

The non-solvent induced phase separation is a method of manufacturing a separation membrane by dissolving a polymer in a solvent and then immersing it in a non-solvent. This method may ease the manufacture of a separation membrane, may lower manufacture cost, and may be applied for the manufacture of various separation membranes.

As explained, since the polymer may be prepared with a relatively high molecular weight, it may help realize a higher strength for a separation membrane. Thus, the polymer is suitable for manufacturing a separation membrane by non-solvent induced phase separation.

The substrate may be a glass plate or a polyester non-woven fabric, but is not limited thereto.

The casting of the polymer solution on a substrate may include casting the polymer solution on the substrate to a thickness of about 25 μm to about 300 μm. The thickness range may be appropriately controlled according to the objective usage which the separation membrane is applied.

The pore forming agent may include polyvinylpyrrolidone, polyethylene glycol, polyethyloxazoline, glycerol, ethylene glycol, diethylene glycol, ethanol, methanol, acetone, phosphoric acid, acetic acid, propanoic acid, lithium chloride, lithium nitrate, lithium perchlorate, and a combination thereof, but is not limited thereto.

The organic solvent may include acetone, acetic acid methanol, 1-methoxy-2-propanol, 1,4-dioxane with a low boiling point (boiling point of less than about 120° C.), N-methyl-2-pyrrolidone (NMP), dimethyl acetamide (DMAC), dimethyl formamide (DMF) with a high boiling point (boiling point of about 150° C. to about 300° C.), and a combination thereof, but is not limited thereto.

The non-solvent is a solvent in which the polymer is insoluble. In general, water may be used because it is readily available and advantageous in terms of cost.

The non-solvent and the solvent should be miscible.

The separation membrane may be a microfiltration membrane, an ultrafiltration membrane, a nanofiltration membrane, a reverse osmotic membrane, or a forward osmotic membrane according to its application. The type of the separation membrane may be classified according to the size of the particle(s) to be separated. A method of manufacturing the separation membrane is not specifically limited, and it may be manufactured by any known method while controlling the size, structure of pores, and the like.

Various kinds of separation membranes may be manufactured and used for various water treatment devices. For example, the separation membranes may be used for a forward osmosis type water treatment device, but is not limited thereto.

According to still another non-limiting embodiment, a forward osmosis device is provided. The forward osmosis device may include a feed solution including impurities to be purified; an osmosis draw solution having higher osmotic pressure than the feed solution; a separation membrane positioned so that one side contacts the feed solution and the other side contacts the osmosis draw solution; a recovery system for separating a solute from the osmosis draw solution; and a connector for reintroducing the solute of the osmosis draw solution separated by the recovery system into the osmosis draw solution contacting the separation membrane.

The forward osmosis device may further include a means (e.g., treatment portion) for producing treated water from the rest of the osmosis draw solution including the water that has passed through the semi-permeable separation membrane by osmotic pressure from the feed solution to the osmosis draw solution, from which the draw solute has been separated by the recovery system.

The details of the separation membrane may be as explained above.

As explained above, the separation membrane may be manufactured by non-solvent induced phase separation to include a porous layer of a finger-like structure. The separation membrane may maintain the pore structure during forward osmosis water treatment due to its relatively high strength characteristic. Since the above pore structure has a relatively high porosity characteristic, and the polymer making up the separation membrane has a relatively high hydrophilicity, permeation flux of the membrane may be increased.

As a separation membrane used in a forward osmosis process is more hydrophilic and has a thinner thickness and higher porosity, the permeation flow rate is improved. Therefore, the above-explained separation membrane is suitable for use in the forward osmosis process.

The operation mechanism of the forward osmosis device is as follows. Water in the feed solution to be treated is passed through the membrane and moves to an osmosis draw solution of a higher concentration due to osmotic pressure. The osmosis draw solution including the water from the feed solution moves to a recovery system for the draw solute to be separated, and the residue solution is output to obtain treated water. Further, the separated draw solute is reused (reintroduced into the osmosis draw solution) so as to contact the feed solution to be treated via the separation membrane.

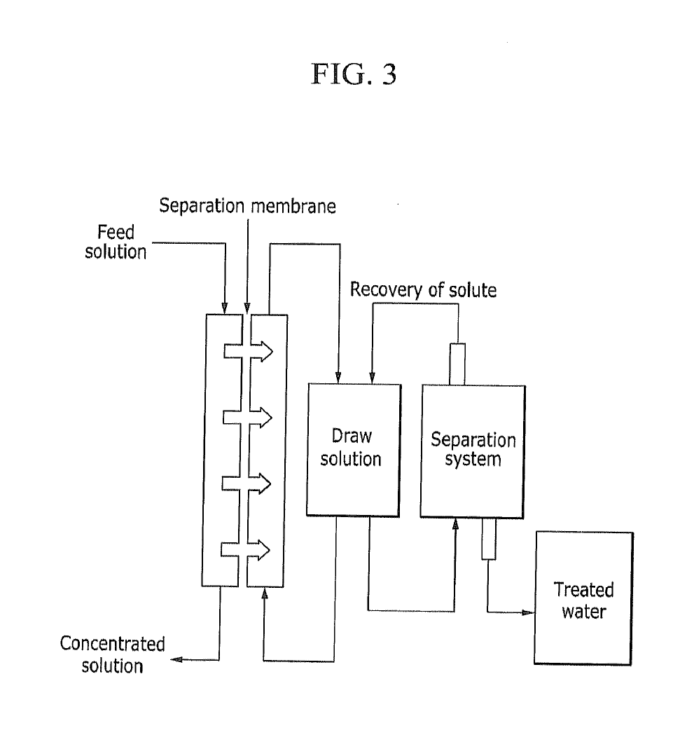

FIG. 3 is a schematic view of a forward osmosis device according to a non-limiting embodiment that is operated according to the above mechanism.

Referring to FIG. 3, the recovery system includes a device for separating a draw solute from the osmosis draw solution.

According to the forward osmosis process, water molecules are moved from a feed solution to an osmosis draw solution having a higher concentration than the feed solution. Then, the draw solute is separated from the osmosis draw solution such that fresh water is produced. The draw solute can be reused by reintroducing it into the osmosis draw solution.

The feed solution may include sea water, brackish water, waste water, tap water for drinking water processing, and the like.

For example, the forward osmosis device may be used for water purification, waste water treatment and reuse, sea water desalination, and the like.

Hereinafter, the non-limiting embodiments are illustrated in more detail with reference to various examples.

### EXAMPLE

### Example 1

### Preparation of Esterified Cellulose Ether Polymer

About 70 g of hydroxypropyl methyl cellulose, about 1120 g of acetic acid anhydride, and about 350 g of pyridine are introduced in a 3 L reactor equipped with an agitator, and then the mixture is agitated at about 200 rpm and reacted at about 90° C. for about 3 hours to prepare acetylated cellulose ether. Herein, pyridine is used as a catalyst. The above-prepared acetylated cellulose ether has a degree of substitution by a methyl group of about 1.94, a degree of molar substitution by a hydroxypropyl of about 0.25, a degree of substitution by an acetyl group of about 1.15, and a weight average molecular weight of about 280,000.

### Example 2

### Manufacture of Separation Membrane

About 15 wt % of the polymer prepared in Example 1 and about 4 wt % of LiCl as a pore forming agent are mixed with dimethyl acetamide (DMAC) to prepare a polymer solution. The polymer solution is casted on a polyester non-woven fabric to a thickness of about 150 μm. The casted substrate is immersed in a coagulation bath of DI water, at 25° C. Deionized water is dropped to the formed membrane to clean remaining solvent thus manufacturing a separation membrane.

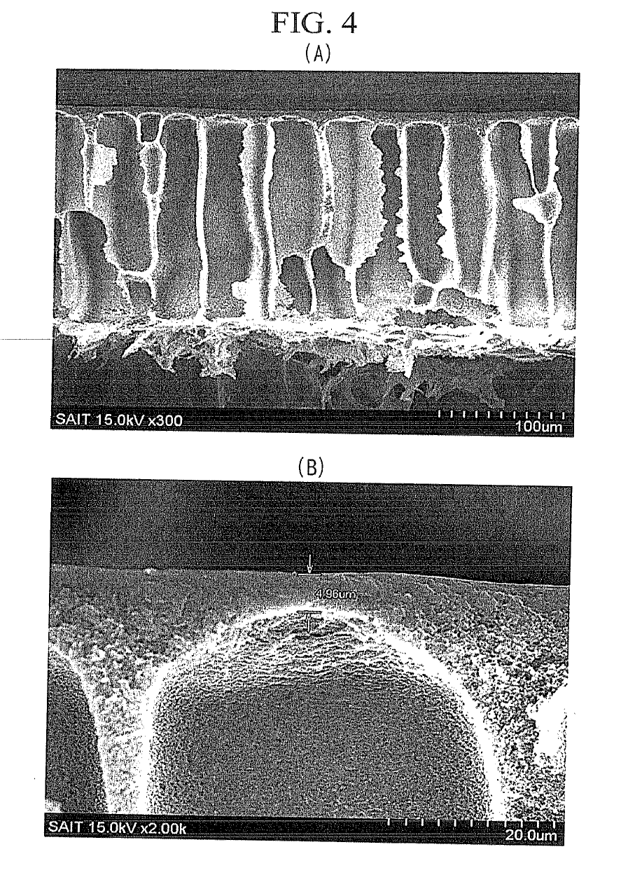

FIGS. 4A-4B are scanning electron microscope (SEM) photographs of the separation membrane prepared according to Example 2.

As result of capillary flow porometer analysis, the average pore size is found to be about 70 nm.

### Comparative Example 1

### Manufacture of a Separation Membrane

A separation membrane is manufactured by the same method as Example 2, except that cellulose triacetate is used instead of the polymer prepared in Example 1 for preparing the polymer solution of Example 2.

### Comparative Example 2

### Membrane Fabrication

A separation membrane is manufactured by the same method as Example 2, except that polyvinylidene fluoride (PVDF) is used instead of the polymer prepared in Example 1 for preparing the polymer solution of Example 2.

Evaluation of Tensile Strength

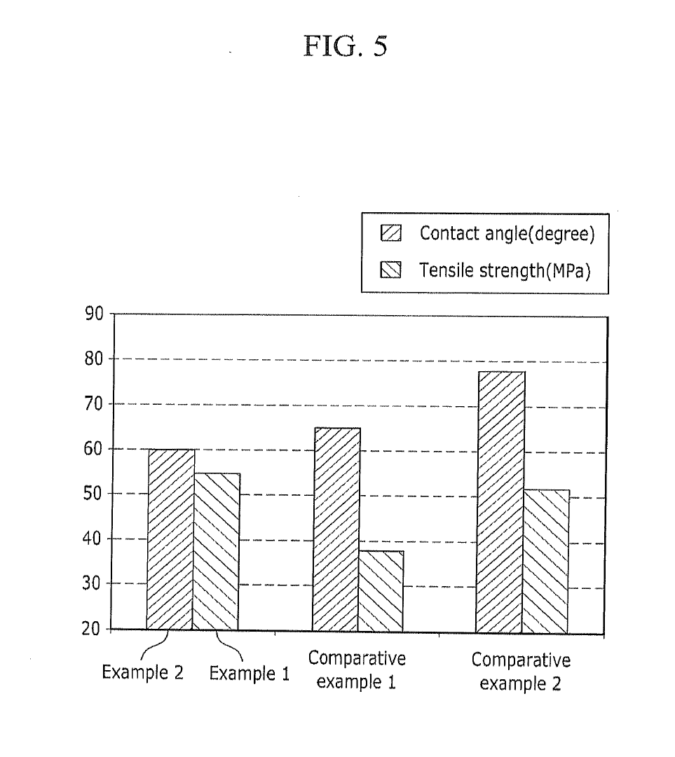

Ten grams each of the polymer prepared in Example 1, cellulose triacetate used in Comparative Example 1, and polyvinylidene fluoride used in Comparative Example 2 are prepared and dissolved in about 90 g of DMF, and then 10 g of the solution is taken to manufacture a film of a thickness of about 0.2 mm. Tensile strength of the film is then measured, and the results are shown in the graph in FIG. 5.

Evaluation of Contact Angle Characteristic

For the membranes of the Example 2 and Comparative Examples 1 and 2, contact angles to water are measured.

The measurement results are shown in the graph in FIG. 5.

Evaluation of Water Permeation Flow Rate Characteristic

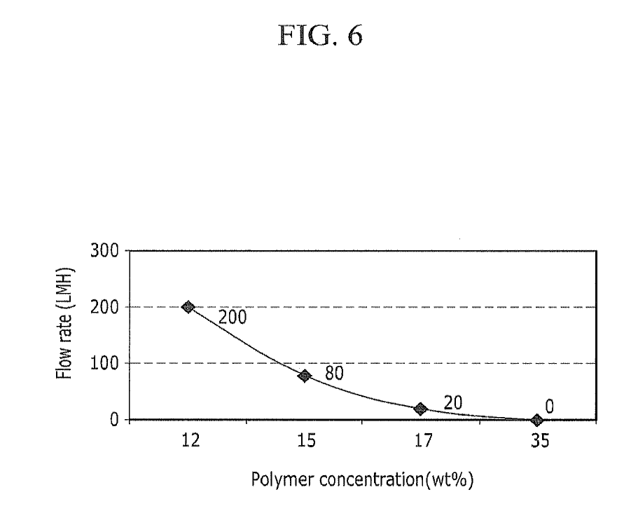

FIG. 6 is a graph showing water permeation flow rates according to a non-limiting embodiment. Referring to FIG. 6, the graph displays the flow rate through a separation membrane as a function of polymer concentration.

Additionally, the water permeation flow rate characteristic of the separation membrane prepared in Example 2 is measured. The separation membrane is consolidated using pure water at about 2 atm for about 2 hours, and then the flow rate is measured at about 1 atm. The results are shown in the following Table 1. The unit of water permeation flow rate is indicated by LMH, wherein LMH denotes the amount of passing water per unit time. Herein, L denotes the amount of water passing the membrane (liter), M denotes the area of the membrane (m²), and H denotes passing time (hour). Thus, it is an evaluation unit indicating how many liters of water pass through a membrane area of 1 m² in 1 hour.

TABLE 1

| | Water permeation flow rate [unit: LMH] |
|---|---|
| Example 2 | 426 |

While this disclosure has been described in connection with various examples, it is to be understood that the disclosure is not limited to the disclosed embodiments. On the contrary, the disclosure is intended to cover various modifications and equivalent arrangements included within the spirit and scope of the appended claims.

### DESCRIPTION OF SYMBOLS

| | |
|---|---|
| 1: substrate | 2: droplet |
| 101: skin layer | 102: porous layer |

What is claimed is:

1. A separation membrane comprising:
at least one polymer including a structural unit represented by the following Chemical Formula 1, 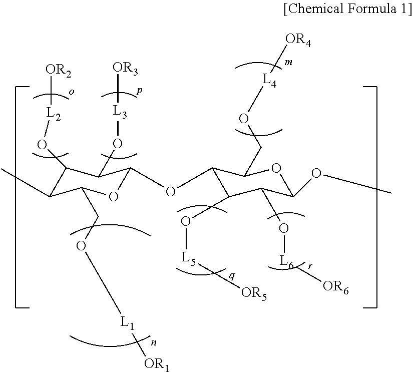
R₁ to R₆ each independently being a hydrogen, a substituted or unsubstituted C1 to C30 alkyl group, a substituted or unsubstituted C3 to C30 cycloalkyl group, a substituted or unsubstituted C2 to C30 heterocycloalkyl group, a substituted or unsubstituted C6 to C30 aryl group, a substituted or unsubstituted C2 to C30 heteroaryl group, a substituted or unsubstituted C7 to C30 alkylaryl group, a substituted or unsubstituted 07 to C30 arylalkyl group, or —COR₇,
R₇ being a substituted or unsubstituted C1 to C30 alkyl group, a substituted or unsubstituted C3 to C30 cycloalkyl group, a substituted or unsubstituted C2 to C30 heterocycloalkyl group, a substituted or unsubstituted C6 to C30 aryl group, a substituted or unsubstituted C2 to C30 heteroaryl group, a substituted or unsubstituted C7 to C30 alkylaryl group, or a substituted or unsubstituted C7 to C30 arylalkyl group,
at least one of R₁ to R₃ and at least one of R₄ to R₆ each independently being the same or different and being —COR₇,
at least one of R₁ to R₃ and at least one of R₄ to R₆ each independently being a substituted or unsubstituted C1 to C30 alkylene group, a substituted or unsubstituted C3 to C30 cycloalkylene group, a substituted or unsubstituted C2 to C30 heterocycloalkylene group, a substituted or unsubstituted C6 to C30 arylene group, a substituted or unsubstituted C2 to C30 heteroarylene group, a substituted or unsubstituted C7 to C30 alkylarylene group, or a substituted or unsubstituted C7 to C30 arylalkylene group,
L₁ to L₆ each independently being a substituted or unsubstituted C1 to C30 alkylene group, a substituted or unsubstituted C3 to C30 cycloalkylene group, a substituted or unsubstituted C2 to C30 heterocycloalkylene group, a substituted or unsubstituted C6 to C30 arylene group, a substituted or unsubstituted C2 to C30 heteroarylene group, a substituted or unsubstituted C7 to C30 alkylarylene group, or a substituted or unsubstituted C7 to C30 arylalkylene group,
n and m each independently being an integer ranging from 0 to 150, the sum of n and m being at least 1, and
o, p, q, and r each independently being an integer ranging from 0 to 100.

2. The separation membrane of claim 1, wherein the at least one polymer has a first degree of substitution (DS) by R₁ to R₆ of an alkyl group, a cycloalkyl group, a heterocycloalkyl group, an aryl group, a heteroaryl group, an alkylaryl group, or an arylalkyl group of about 1 to about 2 per anhydrous glucose unit, and a second degree of substitution by R₁ to R₆ of —COR₇ of about 1 to about 2 per anhydrous glucose unit.

3. The separation membrane of claim 1, wherein the at least one polymer has a weight average molecular weight of about 20,000 to about 800,000.

4. The separation membrane of claim 1, wherein a surface of the separation membrane has a property that yields a contact angle of about 50° to about 65° with regard to water.

5. The separation membrane of claim 1, wherein the separation membrane is a single membrane including a skin layer and a porous layer, the skin layer having a higher density than the porous layer.

6. The separation membrane of claim 5, wherein the skin layer has a thickness of about 0.1 μm to about 10 μm, and a ratio of the thickness of the skin layer to that of the porous layer is about 0.001 to about 0.1.

7. The separation membrane of claim 1, wherein the separation membrane is insoluble in water, and soluble in an organic solvent selected from acetone, acetic acid, methanol, isopropanol, 1-methoxy-2-propanol, trifluoroacetic acid (TFA), tetrahydrofuran (THF), pyridine, methylene chloride, dimethyl formamide (DMF), dimethyl acetamide (DMAC), N-methyl-2-pyrrolidone (NMP), terpineol, 2-butoxyethylacetate, 2-(2-butoxyethoxy)ethylacetate, and a combination thereof.

8. The separation membrane of claim 1, wherein the separation membrane is a microfiltration membrane, an ultrafiltration membrane, a nanofiltration membrane, a reverse osmotic membrane, or a forward osmotic membrane.

9. A forward osmosis device comprising:
a feed solution including impurities;
an osmosis draw solution having a higher osmotic pressure than the feed solution, the osmosis draw solution including an upstream portion and a downstream portion;
the separation membrane according to claim 1, the separation membrane positioned so that a first side contacts the feed solution and an opposing second side contacts the upstream portion of the osmosis draw solution;
a recovery system configured to separate a draw solute from the downstream portion of the osmosis draw solution; and
a connector configured to reintroduce the draw solute from the recovery system into the upstream portion of the osmosis draw solution contacting the separation membrane.

10. The forward osmosis device of claim 9, further comprising:
a treatment portion arranged downstream from the recovery system, the treatment portion configured to produce treated water from the downstream portion of the osmosis draw solution, the downstream portion of the osmosis draw solution including water from the feed solution that has passed through the separation membrane to the osmosis draw solution by osmotic pressure, the treated water having had at least the draw solute separated therefrom by the recovery system.

11. A method of preparing a polymer, comprising:
etherifying a cellulose compound to obtain a cellulose ether compound having at least one hydroxyl group, and
esterifying the cellulose ether compound to obtain the polymer, the polymer being an esterified cellulose ether including a structural unit represented by the following Chemical Formula 1, 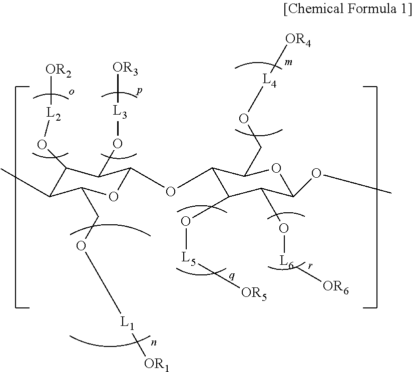
R₁ to R₆ each independently being a hydrogen, a substituted or unsubstituted C1 to C30 alkyl group, a substituted or unsubstituted C3 to C30 cycloalkyl group, a substituted or unsubstituted C2 to C30 heterocycloalkyl group, a substituted or unsubstituted C6 to C30 aryl group, a substituted or unsubstituted C2 to C30 heteroaryl group, a substituted or unsubstituted C7 to C30 alkylaryl group, a substituted or unsubstituted C7 to C30 arylalkyl group, or —COR₇,
R₇ being a substituted or unsubstituted C1 to C30 alkyl group, a substituted or unsubstituted C3 to C30 cycloalkyl group, a substituted or unsubstituted C2 to C30 heterocycloalkyl group, a substituted or unsubstituted C6 to C30 aryl group, a substituted or unsubstituted C2 to C30 heteroaryl group, a substituted or unsubstituted C7 to C30 alkylaryl group, or a substituted or unsubstituted C7 to C30 arylalkyl group,
at least one of R₁ to R₃ and at least one of R₄ to R₆ each independently being the same or different and being —COR₇, and
at least one of R₁ to R₃ and at least one of R₄ to R₆ each independently being a substituted or unsubstituted C1 to C30 alkylene group, a substituted or unsubstituted C3 to C30 cycloalkylene group, a substituted or unsubstituted C2 to C30 heterocycloalkylene group, a substituted or unsubstituted C6 to C30 arylene group, a substituted or unsubstituted C2 to C30 heteroarylene group, a substituted or unsubstituted C7 to C30 alkylarylene group, or a substituted or unsubstituted C7 to C30 arylalkylene group,
L₁ to L₆ each independently being a substituted or unsubstituted C1 to C30 alkylene group, a substituted or unsubstituted C3 to C30 cycloalkylene group, a substituted or unsubstituted C2 to C30 heterocycloalkylene group, a substituted or unsubstituted C6 to C30 arylene group, a substituted or unsubstituted C2 to C30 heteroarylene group, a substituted or unsubstituted C7 to C30 alkylarylene group, or a substituted or unsubstituted C7 to C30 arylalkylene group,
n and m each independently being an integer ranging from 0 to 150, the sum of n and m being at least 1, and
o, p, q, and r each independently being an integer ranging from 0 to 100.

12. The method of claim 11, wherein the etherifying a cellulose compound includes
substituting a hydrogen of at least a first hydroxyl group of the cellulose compound with an alkyl group, a cycloalkyl group, a heterocycloalkyl group, an aryl group, a heteroaryl group, an alkylaryl group, or an arylalkyl group to form an ether group; and
substituting a hydrogen of at least a second hydroxyl group of the cellulose compound with an alkyl group, a cycloalkyl group, a heterocycloalkyl group, an aryl group, a heteroaryl group, an alkylaryl group, or an arylalkyl group that includes at least a third hydroxyl group.

13. The method of claim 12, further comprising:
repeatedly substituting a hydrogen of the third hydroxyl group with an alkyl group, a cycloalkyl group, a heterocycloalkyl group, an aryl group, a heteroaryl group, an alkylaryl group, or an arylalkyl group that includes at least a fourth hydroxyl group so as to form a moiety of —(O-L₁)ₙ-, —(O-L₂)ₒ-, —(O-L₃)ₚ-, —(O-L₄)ₘ-, —(O-L₅)_{q}-, or —(O-L₆)ᵣ- in the structure of Chemical Formula 1.

14. The method of claim 11, wherein the esterifying the cellulose ether compound includes substituting a hydrogen of the at least one hydroxyl group of the cellulose ether compound with a —COR₇ group.

15. The method of claim 11, wherein the esterifying the cellulose ether compound is performed such that the polymer has a weight average molecular weight of about 20,000 to about 800,000.

16. A method of manufacturing a separation membrane, comprising
preparing a polymer solution including at least one polymer including a structural unit represented by the following Chemical Formula 1, and an organic solvent;
casting the polymer solution on a substrate; and
immersing the substrate with the polymer solution in a non-solvent to form a skin layer and a porous layer, 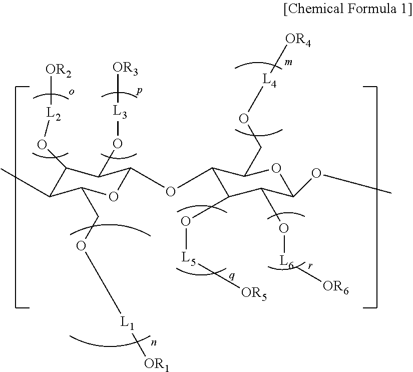
R₁ to R₆ each independently being a hydrogen, a substituted or unsubstituted C1 to C30 alkyl group, a substituted or unsubstituted C3 to C30 cycloalkyl group, a substituted or unsubstituted C2 to C30 heterocycloalkyl group, a substituted or unsubstituted C6 to C30 aryl group, a substituted or unsubstituted C2 to C30 heteroaryl group, a substituted or unsubstituted C7 to C30 alkylaryl group, a substituted or unsubstituted C7 to C30 arylalkyl group, or —COR₇,
R₇ being a substituted or unsubstituted C1 to C30 alkyl group, a substituted or unsubstituted C3 to C30 cycloalkyl group, a substituted or unsubstituted C2 to C30 heterocycloalkyl group, a substituted or unsubstituted C6 to C30 aryl group, a substituted or unsubstituted C2 to C30 heteroaryl group, a substituted or unsubstituted C7 to C30 alkylaryl group, or a substituted or unsubstituted C7 to C30 arylalkyl group,
at least one of R₁ to R₃ and at least one of R₄ to R₆ each independently being the same or different and being —COR₇, and
at least one of R₁ to R₃ and at least one of R₄ to R₆ each independently being a substituted or unsubstituted C1 to C30 alkylene group, a substituted or unsubstituted C3 to C30 cycloalkylene group, a substituted or unsubstituted C2 to C30 heterocycloalkylene group, a substituted or unsubstituted C6 to C30 arylene group, a substituted or unsubstituted C2 to C30 heteroarylene group, a substituted or unsubstituted C7 to C30 alkylarylene group, or a substituted or unsubstituted C7 to C30 arylalkylene group,
L₁ to L₆ each independently being a substituted or unsubstituted C1 to C30 alkylene group, a substituted or unsubstituted C3 to C30 cycloalkylene group, a substituted or unsubstituted C2 to C30 heterocycloalkylene group, a substituted or unsubstituted C6 to C30 arylene group, a substituted or unsubstituted C2 to C30 heteroarylene group, a substituted or unsubstituted C7 to C30 alkylarylene group, or a substituted or unsubstituted C7 to C30 arylalkylene group,
n and m each independently being an integer ranging from 0 to 150, the sum of n and m being at least 1, and
o, p, q, and r each independently being an integer ranging from 0 to 100.

17. The method of claim 16, wherein the preparing a polymer solution includes combining about 5 to about 30 wt % of the at least one polymer, about 0 to about 10 wt % of a pore forming agent, and about 50 to about 95 wt % of the organic solvent.

18. The method of claim 17, wherein the preparing a polymer solution further includes ensuring that the pore forming agent includes polyvinylpyrrolidone, polyethylene glycol, polyethyloxazoline, glycerol, ethylene glycol, diethylene glycol, ethanol, methanol, acetone, phosphoric acid, acetic acid, propanoic acid, lithium chloride, lithium nitrate, lithium perchlorate, or a combination thereof.

19. The method of claim 16, wherein the casting includes introducing the polymer solution onto the substrate, the substrate being a glass plate or a polyester non-woven fabric.

20. The method of claim 16, wherein the casting includes introducing the polymer solution onto the substrate to a thickness of about 25 μm to about 300 μm.

21. The method of claim 16, wherein the preparing a polymer solution includes ensuring that the organic solvent includes acetone, acetic acid methanol, 1-methoxy-2-propanol, 1,4-dioxane with a boiling point of less than about 120° C., N-methyl-2-pyrrolidone (NMP), dimethyl acetamide (DMAC), dimethyl formamide (DMF) with a boiling point of about 150° C. to about 300° C., or a combination thereof.
